(19) United States
(12) Patent Application Publication
(10) Pub. No.: US 2020/0360647 A1
(43) Pub. Date: Nov. 19, 2020

(54) Use Of Inhaled Nitric Oxide For The Improvement Of Right And/Or Left Ventricular Function

(71) Applicant: Bellerophon Pulse Technologies LLC, Warren, NJ (US)

(72) Inventors: Deborah Quinn, Morristown, NJ (US); Parag Shah, Morristown, NJ (US)

(73) Assignee: Bellerophon Pulse Technologies LLC, Warren, NJ (US)

(21) Appl. No.: 16/643,167

(22) PCT Filed: Aug. 29, 2018
(86) PCT No.: PCT/US18/48524

§ 371 Date: Feb. 28, 2020

Related U.S. Application Data
(60) Provisional application No. 62/611,325, filed on Dec. 28, 2017.
(60) Provisional application No. 62/552,022, filed on Aug. 30, 2017.

(51) Int. Cl. A61M 16/12; A61M 16/00; A61K 33/00; A61P 11/00

(52) U.S. Cl. CPC A61M 16/12; A61M 16/0003; A61K 33/00; A61P 11/00; A61M 2202/0275; A61M 2230/30; A61M 2016/0027; A61M 2210/1039; A61M 2210/125; A61M 2230/40; A61M 2202/0208

(57) ABSTRACT

Described herein are methods of using inhaled nitric oxide for improving and/or maintaining right ventricular function and/or left ventricular function. Some methods relate to the long-term administration of inhaled nitric oxide to a patient with pulmonary hypertension.

### CROSS-REFERENCE TO RELATED APPLICATIONS

This application claims priority to U.S. Provisional Application Ser. No. 62/552,022 filed on Aug. 30, 2017 and U.S. Provisional Application Ser. No. 62/611,325 filed Dec. 28, 2017, which are incorporated by reference herein in their entirety.

### TECHNICAL FIELD

Principles and embodiments of the present invention generally relate to the field of inhaled nitric oxide delivery.

### BACKGROUND

A human heart has four chambers that work together to pump blood through a person's circulatory system. Of these, two chambers are known as ventricles, with the left ventricle (LV) pumping blood to the systemic circulation and the right ventricle (RV) pumping blood into the pulmonary circulation.

The importance of proper RV function in maintaining normal cardiac output is well established. One role of the RV is maintenance of adequate pulmonary perfusion pressure under varying circulatory and loading conditions in order to deliver desaturated venous blood to the gas exchange membranes of the lung. Another role of the RV is the maintenance of a low systemic venous pressure to prevent tissue and organ congestion.

In addition, RV function has been shown to be a major determinant of clinical outcome and has the potential to be predictive of outcomes in clinical research and therapy. Moreover, due to interdependencies of the RV and the LV, improvements in RV function can also lead to improvements in LV function.

Accordingly, there is a need for new therapies to improve RV function and/or LV function.

### SUMMARY

One aspect of the present invention pertains to a method of maintaining or improving RV function using inhaled nitric oxide (iNO). In various embodiments of this aspect, the method comprising administering to a patient in need thereof an effective amount of iNO for at least 2 days.

In one or more embodiments, the patient has pulmonary hypertension (PH). In one or more embodiments, the PH comprises one or more of pulmonary arterial hypertension (PAH) (WHO Group I), PH associated with left heart disease (WHO Group 2), PH associated with lung disease and/or chronic hypoxemia (WHO Group 3), chronic thromboembolic pulmonary hypertension (WHO Group 4) or PH with unclear multifactorial mechanisms (WHO Group 5).

In one or more embodiments, the patient has a low, intermediate, or high probability of PH.

In one or more embodiments, the patient has PAH.

In one or more embodiments, the patient has WHO Group 3 PH associated with idiopathic pulmonary fibrosis (PH-IPF) or WHO Group 3 PH associated with chronic obstructive pulmonary disease (PH-COPD).

In one or more embodiments, the patient has PH associated with pulmonary edema from high altitude sickness.

In one or more embodiments, the patient has PH associated with sarcoidosis.

In one or more embodiments, the patient has been placed on a lung transplant waiting list.

In one or more embodiments, the patient has received a lung transplant.

In one or more embodiments, the patient has a ventilation-perfusion (V/Q) mismatch.

In one or more embodiments, the iNO is administered for at least 1 week.

In one or more embodiments, the iNO is administered for at least 2 weeks.

In one or more embodiments, the iNO is administered for at least 4 weeks.

In one or more embodiments, the iNO is administered for at least 3 months.

In one or more embodiments, the iNO is administered for at least 6 hours a day.

In one or more embodiments, the iNO is administered for at least 12 hours a day.

In one or more embodiments, the effective amount of iNO is in the range of about 5 to about 300 micrograms NO per kilogram ideal body weight per hour (mcg/kg IBW/hr).

In one or more embodiments, the effective amount is in the range of about 30 to about 75 mcg/kg IBW/hr.

In one or more embodiments, maintaining or improving RV function comprises maintaining or improving one or more of the following parameters: RV fractional area change (RVFAC), tricuspid annular motion, tricuspid annular plane systolic excursion (TAPSE), systolic pulmonary artery pressure (sPAP), tricuspid annular systolic velocity (TASV), and Tei index.

In one or more embodiments, the administration of iNO provides an average increase in TAPSE in a group of patients after 4 weeks of iNO administration of at least 1 millimeter (mm).

In one or more embodiments, the administration of iNO provides an average increase in TAPSE in a group of patients after 4 weeks of iNO administration of at least 2 mm.

In one or more embodiments, the administration of iNO provides an average increase in TAPSE in a group of patients after 4 weeks of iNO administration of at least 5%.

In one or more embodiments, the administration of iNO provides an average increase in TAPSE in a group of patients after 4 weeks of iNO administration of at least 10%.

Another aspect of the present invention pertains to a method of maintaining or improving LV function using iNO. In various embodiments of this aspect, the method comprising administering to a patient in need thereof an effective amount of iNO for at least 2 days.

In one or more embodiments, the patient has PH. In one or more embodiments, the PH comprises one or more of PAH, PH associated with left heart disease, PH associated with lung disease and/or chronic hypoxemia, chronic thromboembolic pulmonary hypertension or PH with unclear multifactorial mechanisms.

In one or more embodiments, the patient has a low, intermediate, or high probability of PH.

In one or more embodiments, the patient has PAH.

In one or more embodiments, the patient has PH-IPF or PH-COPD.

In one or more embodiments, the patient has been placed on a lung transplant waiting list.

In one or more embodiments, the patient has received a lung transplant.

In one or more embodiments, the patient has a V/Q mismatch.

In one or more embodiments, the iNO is administered for at least 1 week.

In one or more embodiments, the iNO is administered for at least 2 weeks.

In one or more embodiments, the iNO is administered for at least 4 weeks.

In one or more embodiments, the iNO is administered for at least 3 months.

In one or more embodiments, the iNO is administered for at least 6 hours a day.

In one or more embodiments, the iNO is administered for at least 12 hours a day.

In one or more embodiments, the effective amount of iNO is in the range of about 5 to about 300 mcg/kg IBW/hr.

In one or more embodiments, the effective amount is in the range of about 30 to about 75 mcg/kg IBW/hr.

In one or more embodiments, maintaining or improving LV function comprises maintaining or improving one or more of the following parameters: LV ejection fraction (LVEF), LV size, and LV early diastolic relaxation velocity.

In one or more embodiments, a plurality of pulses of a gas comprising NO is administered to the patient over a plurality of breaths.

In one or more embodiments, the gas comprising NO is not administered to the patient in at least one breath of the plurality of breaths.

In one or more embodiments, the maximum time period between successive pulses of the gas comprising NO does not exceed about 30, about 25, about 20, about 15, about 14, about 13, about 12, about 11, about 10, about 9, about 8.5, about 8, about 7.5, about 7, about 6.5 or about 6 seconds.

In one or more embodiments, the maximum number of consecutive skipped breaths does not exceed three, two or one breaths.

In one or more embodiments, the average time period between successive pulses of the gas comprising NO does not exceed about 25, about 20, about 15, about 14, about 13, about 12, about 11, about 10, about 9, about 8.5, about 8, about 7.5, about 7, about 6.5 or about 6 seconds.

In one or more embodiments, the average time period between successive pulses of the gas comprising NO does not exceed about 3, about 2.5, about 2, about 1.5 or about 1 breaths.

In one or more embodiments, at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 700, about 750, about 800, about 850, about 900, about 950 or about 1,000 pulses of the gas comprising NO is administered to the patient every hour.

Another aspect of the present invention pertains to a method of using iNO to maintain or improve cardiac function in a patient on a lung transplant waiting list. In various embodiments of this aspect, the method comprises administering to the patient an effective amount of iNO for at least 2 days.

In one or more embodiments, the patient has PH. In one or more embodiments, the PH comprises one or more of PAH, PH associated with left heart disease, PH associated with lung disease and/or chronic hypoxemia, chronic thromboembolic pulmonary hypertension or PH with unclear multifactorial mechanisms.

In one or more embodiments, the patient has PAH.

In one or more embodiments, the patient has PH-IPF or PH-COPD.

In one or more embodiments, the patient has PH associated with pulmonary edema from high altitude sickness.

In one or more embodiments, the patient has PH associated with sarcoidosis.

In one or more embodiments, the patient has a V/Q mismatch.

In one or more embodiments, the iNO is administered for at least 1 week.

In one or more embodiments, the iNO is administered for at least 2 weeks.

In one or more embodiments, the iNO is administered for at least 4 weeks.

In one or more embodiments, the iNO is administered for at least 3 months.

In one or more embodiments, the iNO is administered for at least 6 hours a day.

In one or more embodiments, the iNO is administered for at least 12 hours a day.

In one or more embodiments, the effective amount of iNO is in the range of about 5 to about 300 mcg/kg IBW/hr.

In one or more embodiments, the effective amount is in the range of about 30 to about 75 mcg/kg IBW/hr.

In one or more embodiments, maintaining or improving cardiac function comprises one or more of (i) maintaining or improving RV function or (ii) maintaining or improving LV function.

In one or more embodiments, maintaining or improving RV function comprises maintaining or improving one or more of the following parameters: RVFAC, sPAP, tricuspid annular motion, TAPSE, TASV, and Tei index.

In one or more embodiments, the administration of iNO provides an average increase in TAPSE in a group of patients after 4 weeks of iNO administration of at least 1 mm.

In one or more embodiments, the administration of iNO provides an average increase in TAPSE in a group of patients after 4 weeks of iNO administration of at least 2 mm.

In one or more embodiments, the administration of iNO provides an average increase in TAPSE in a group of patients after 4 weeks of iNO administration of at least 5%.

In one or more embodiments, the administration of iNO provides an average increase in TAPSE in a group of patients after 4 weeks of iNO administration of at least 10%.

In one or more embodiments, maintaining or improving LV function comprises maintaining or improving one or more of the following parameters: LVEF, LV size, and LV early diastolic relaxation velocity.

In one or more embodiments, a plurality of pulses of a gas comprising NO is administered to the patient over a plurality of breaths.

In one or more embodiments, the gas comprising NO is not administered to the patient in at least one breath of the plurality of breaths.

In one or more embodiments, the maximum time period between successive pulses of the gas comprising NO does not exceed about 30, about 25, about 20, about 15, about 14, about 13, about 12, about 11, about 10, about 9, about 8.5, about 8, about 7.5, about 7, about 6.5 or about 6 seconds.

In one or more embodiments, the maximum number of consecutive skipped breaths does not exceed three, two or one breaths.

In one or more embodiments, the average time period between successive pulses of the gas comprising NO does not exceed about 25, about 20, about 15, about 14, about 13, about 12, about 11, about 10, about 9, about 8.5, about 8, about 7.5, about 7, about 6.5 or about 6 seconds.

In one or more embodiments, the average time period between successive pulses of the gas comprising NO does not exceed about 3, about 2.5, about 2, about 1.5 or about 1 breaths.

In one or more embodiments, at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 700, about 750, about 800, about 850, about 900, about 950 or about 1,000 pulses of the gas comprising NO is administered to the patient every hour.

### BRIEF DESCRIPTION OF THE DRAWINGS

Further features of the present invention will become apparent from the following written description and the accompanying figures, in which:

FIG. 1 shows the treatment visit schedule for Part 2a of a three-part clinical trial evaluating the use of iNO;

FIG. 2 shows the treatment visit schedule for Part 2b of a three-part clinical trial evaluating the use of iNO;

FIG. 3 shows the treatment visit dose titration details for Part 3a of a three-part clinical trial evaluating the use of iNO;

FIG. 4 shows the treatment visit schedule for Part 3b of a three-part clinical trial evaluating the use of iNO;

FIG. 5 shows the change in six-minute walk distance (6MWD) in PH-COPD patients at baseline and during chronic iNO therapy;

FIG. 6 shows sPAP in PH-COPD patients at baseline, during chronic iNO therapy and after discontinuation of chronic iNO therapy; and

FIG. 7 shows TAPSE in PH-COPD patients at baseline, during chronic iNO therapy and after discontinuation of chronic iNO therapy.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

It has surprisingly been discovered that long-term iNO therapy maintains and/or improves RV function in patients with PH. Although short-term iNO therapy has been used in the management of RV failure in heart transplant recipients, long-term iNO therapy has never been shown to maintain or improve RV function. Accordingly, various embodiments of the present invention relate to maintaining and/or improving RV function using long-term iNO therapy.

Maintenance and/or improvements in RV function can be assessed by many echocardiographic measurements. One such quantitative approach to assess RV function is the measurement of the TAPSE. The TAPSE estimates RV systolic function by measuring the level of systolic excursion of the lateral tricuspid valve annulus towards the apex. An excellent correlation between the TAPSE and RV ejection fraction as assessed by radionuclide angiography has previously been established and the approach appears reproducible and proven to be a strong predictor of prognosis in heart failure. [Reference: Heart. 2006 April; 92(Suppl 1): i19-i26.]

Other echocardiographic measurements that may be used to assess maintenance and/or improvements in RV function include, but are not limited to, RVFAC, sPAP, tricuspid annular motion, TAPSE, TASV, and Tei index.

Accordingly, in one or more embodiments, the iNO therapy maintains or improves one or more of the following parameters: TAPSE, RVFAC, sPAP, tricuspid annular motion, TAPSE, TASV, and Tei index. In some embodiments, maintenance of a parameter corresponds to no change in that parameter over a certain time period. In some embodiments, if a parameter is expected to worsen in an untreated patient over time (e.g. TAPSE is expected to decrease in untreated PH patients), then maintenance of a parameter also includes a clinical worsening of the parameter that is a smaller magnitude than the clinical worsening that is expected for an untreated patient.

In one or more embodiments, the iNO therapy maintains or increases TAPSE over a certain time period, such as after administering iNO for 2, 3, 4, 5, 6, 7, 8, 9, 10, 15, 20, 25, 30 days 1, 2, 3, 4, 5, 6, 7 or 8 weeks or 1, 2, 3, 4, 5, 6, 7, 8, 9, 10, 12, 18 or 24 months or at least 1, 2, 3, 4 or 5 years.

In one or more embodiments, the patient's TAPSE does not change during iNO therapy, even though the TAPSE is expected to decrease in an untreated patient. In other embodiments, a patient's TAPSE is increased over a certain time period. Exemplary increases in TAPSE include increases of about 1, about 2, about 3, about 4, about 5, about 6, about 7, about 8, about 9 or about 10 mm. Exemplary increases in TAPSE can also be expressed in percentages, such as increases of about 5, about 10, about 15, about 20, about 25, about 30, about 35, about 40, about 45, about 50, about 55, about 60, about 65 or about 70%.

In one or more embodiments, 1 week of iNO therapy provides an average increase in TAPSE in a group of patients of at least 1 mm. In various embodiments, the average increase in TAPSE in the group of patients after 1 week of iNO therapy is at least about 1, about 2, about 3, about 4, about 5, about 6, about 7, about 8, about 9 or about 10 mm.

In one or more embodiments, 1 week of iNO therapy provides an average increase in TAPSE in a group of patients of at least 5%. In various embodiments, the average increase in TAPSE in the group of patients after 1 week of iNO therapy is at least about 5, about 10, about 15, about 20, about 25, about 30, about 35, about 40, about 45, about 50, about 55, about 60, about 65 or about 70%.

In one or more embodiments, 2 weeks of iNO therapy provides an average increase in TAPSE in a group of patients of at least 1 mm. In various embodiments, the average increase in TAPSE in the group of patients after 2 weeks of iNO therapy is at least about 1, about 2, about 3, about 4, about 5, about 6, about 7, about 8, about 9 or about 10 mm.

In one or more embodiments, 2 weeks of iNO therapy provides an average increase in TAPSE in a group of patients of at least 5%. In various embodiments, the average increase in TAPSE in the group of patients after 2 weeks of iNO therapy is at least about 5, about 10, about 15, about 20, about 25, about 30, about 35, about 40, about 45, about 50, about 55, about 60, about 65 or about 70%.

In one or more embodiments, 4 weeks of iNO therapy provides an average increase in TAPSE in a group of patients of at least 1 mm. In various embodiments, the average increase in TAPSE in the group of patients after 4 weeks of iNO therapy is at least about 1, about 2, about 3, about 4, about 5, about 6, about 7, about 8, about 9 or about 10 mm.

In one or more embodiments, 4 weeks of iNO therapy provides an average increase in TAPSE in a group of patients of at least 5%. In various embodiments, the average increase in TAPSE in the group of patients after 4 weeks of iNO therapy is at least about 5, about 10, about 15, about 20, about 25, about 30, about 35, about 40, about 45, about 50, about 55, about 60, about 65 or about 70%.

As described above, due to the interdependencies of RV function and LV function, improving RV function can also improve LV function. Thus, iNO therapy can also be used to maintain and/or improve LV function in a patient.

Maintenance and/or improvements in LV function can be assessed by many echocardiographic measurements. Echocardiographic measurements that may be used to assess maintenance and/or improvements in LV function include, but are not limited to, LVEF, LV size, and LV early diastolic relaxation velocity.

Accordingly, in one or more embodiments, the iNO therapy maintains or improves one or more of the following parameters: LVEF, LV size, and LV early diastolic relaxation velocity. As described above, in some embodiments, maintenance of a parameter corresponds to no change in that parameter over a certain time period. In some embodiments, if a parameter is expected to worsen in an untreated patient over time, then maintenance of a parameter also includes a clinical worsening of the parameter that is a smaller magnitude than the clinical worsening that is expected for an untreated patient.

In one or more embodiments, the patient or group of patients are diagnosed with PH. The patient(s) can be diagnosed by a cardiologist, pulmonologist or other physician according to suitable criteria using techniques such as echocardiography, right heart catheterization (RHC), etc. Examples of such criteria include, but are not limited to, patients that have a mean pulmonary arterial pressure (mPAP) at rest of at least 25 mm Hg, or a tricuspid regurgitation velocity greater than 2.9 m/s, or other combinations of factors as determined by an appropriate physician. The World Health Organization (WHO) has defined five categories of PH: PAH (WHO Group 1); PH associated with left heart disease (WHO Group 2), PH associated with lung disease and/or chronic hypoxemia (WHO Group 3), chronic thromboembolic pulmonary hypertension (WHO Group 4) or PH with unclear multifactorial mechanisms (WHO Group 5).

Examples of WHO Group 2 patients include those with systolic dysfunction, diastolic dysfunction and/or valvular disease.

Examples of WHO Group 3 patients include PH-COPD patients and those with interstitial lung disease (ILD) such as PH-IPF patients. Other examples of WHO Group 3 patients include those with combined pulmonary fibrosis and emphysema (CPFE), chronic high altitude exposure, or other lung diseases such as sleep disordered breathing or developmental diseases. COPD, ILD and other lung diseases can be diagnosed according to any suitable factor or combination of factors, such as those set forth in the guidelines of the American Thoracic Society. One exemplary set of criteria for diagnosing COPD is the Global initiative for chronic Obstructive Lung Disease (GOLD) criteria. In at least one embodiment, the patient has PH-COPD. In at least one embodiment, the patient has PH and ILD, such as a patient with PH-IPF. In at least one embodiment, the patient has PH associated with pulmonary edema from high altitude sickness.

In one or more embodiments, the patient has a V/Q mismatch.

Examples of WHO Group 5 patients include those with hematologic disorders, systemic disorders that have lung involvement (e.g. sarcoidosis, Langerhans cell histiocytosis, lymphangioleiomyomatosis, neurofibromatosis and vasculitis), metabolic disorders (e.g. thyroid disorders and glycogen storage disease), and other diseases such as tumor obstruction or renal failure. In at least one embodiment, the patient has PH associated with sarcoidosis.

In one or more embodiments, the patient or group of patients has a low, intermediate, or high probability of PH as determined by echocardiography or other suitable technique. One exemplary set of criteria for evaluating the probability of PH is set forth in the 2015 ESC/ERS Guidelines for Diagnosis and Treatment of Pulmonary Hypertension. In at least one embodiment, the patient has a low echocardiographic probability of PH. In at least one embodiment, the patient has an intermediate echocardiographic probability of PH. In at least one embodiment, the patient has a high echocardiographic probability of PH.

In one or more embodiments, the patient has been placed on a lung transplant waiting list, and the iNO therapy is used to maintain or improve RV and/or LV function before the lung transplant. In other embodiments, the patient has already received a lung transplant.

Patients in need of a lung transplant are evaluated and receive a lung allocation score (LAS), which estimates the severity of each candidates' illness and his or her chance of success following a lung transplant. Those with a higher LAS receive a higher priority for a lung offer when a compatible lung becomes available. Improving or maintaining cardiac function (e.g. RV and/or LV function) improves the likelihood that a patient will survive long enough to receive a lung transplant. Moreover, improving or maintaining cardiac function (e.g. RV and/or LV function) improves a patient's prognosis following lung transplant. Accordingly, in one or more embodiments, iNO therapy can be provided to patients on a lung transplant list, particularly patients on a lung transplant list that have PH. Also, in one or more embodiments, iNO therapy may influence one or more factors used to determine the patient's LAS, and thus the iNO therapy may change the patient's LAS.

The iNO may be administered continuously, or by a series of pulses, or any other suitable technique for delivering iNO to a patient's lungs. Exemplary devices for the administration of iNO are described in U.S. Pat. Nos. 5,558,083; 7,523,752; 8,757,148; 8,770,199; 8,893,717; 8,944,051; U.S. Pat. App. Pub. No. 2013/0239963; U.S. Pat. App. Pub. No. 2014/0000596; and U.S. Pat. App. Pub. No. 2016/0106949, the disclosures of which are hereby incorporated by reference in their entireties.

In one or more embodiments, iNO is administered by a NO delivery device utilizing cylinders containing NO and a carrier gas such as nitrogen (N₂). Exemplary NO cylinder concentrations include, but are not limited to, concentrations in the range of about 100 ppm to about 15,000 ppm, such as about 100, about 200, about 300, about 400, about 500, about 600, about 700, about 800, about 900, about 1,000, about 1,500, about 2,000, about 2,500, about 3,000, about 3,500, about 4,000, about 4,500, about 5,000, about 6,000, about 7,000, about 8,000, about 9,000, about 10,000 or about 15,000 ppm. In one or more embodiments, the NO cylinder concentration is about 4,880 ppm.

In one or more embodiments, the NO is generated bedside or at the point of administration. For example, various chemical reactions can be used to generate NO, such reacting N₂ and oxygen (O₂) in the presence of an electrode, or reacting nitrogen dioxide (NO₂) with a reducing agent.

In one or more embodiments, the iNO is administered as a series of pulses. The iNO may have a specific pulse volume, such as about 0.1, about 0.2, about 0.3, about 0.4, about 0.5, about 0.6, about 0.7, about 0.8, about 0.9, about 1, about 1.5, about 2, about 3, about 4 or about 5 mL. The pulse volume may be the same from one breath to the next, or the pulse volume may vary according to the patient's breathing rate and/or the amount of iNO already delivered to the patient.

In one or more embodiments, the effective amount of iNO is in the range of about 5 to about 300 mcg/kg IBW/hr. A patient's ideal body weight correlates with the patient's estimated lung size, and is a function of the patient's sex and height. In various embodiments, the dose of iNO is about 5, about 10, about 15, about 20, about 25, about 30, about 35, about 40, about 45, about 50, about 55, about 60, about 65 or about 70 mcg/kg IBW/hr.

In one or more embodiments, a constant dose of iNO is delivered to the patient in each breath, such as a constant dose in nmol/breath, ng/breath or mL/breath. Exemplary doses include about 10, about 20, about 30, about 40, about 50, about 60, about 70, about 80, about 90, about 100, about 150, about 200, about 300, about 400, about 500, about 600, about 700, about 800, about 900, about 1,000 or about 1,500 nmol NO per breath.

In one or more embodiments, the iNO is administered continuously at a constant concentration. For example, the iNO may be administered at a constant concentration of about 1 ppm to about 100 ppm. In various embodiments, the dose of iNO is about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95 or about 100 ppm.

In one or more embodiments, a desired quantity of gas is administered to the patient over a plurality of breaths in a way that is independent of the patient's respiratory pattern. For example, a patient's iNO dose may be prescribed in terms of mcg/kg IBW/hr, such that a desired amount is delivered to the patient every hour regardless of the patient's respiratory pattern or breathing rate. The NO delivery device may have an input such as a dial, display, touchscreen or other user interface to receive the patient's prescription. An amount of NO per breath (e.g. nmol NO, ng NO, mL of gas comprising NO, etc.) can be calculated based on the patient's current respiratory pattern, and that amount of NO can be delivered to the patient in the next breath or for several breaths. The NO delivery device may monitor the patient's respiratory pattern or breathing rate (or changes in the respiratory pattern or breathing rate) and re-calculate and/or otherwise adjust the amount of NO-containing gas that is delivered on the current breath or on subsequent breaths. The NO delivery device can have a control system with appropriate software and/or hardware (e.g. flow sensors, pressure sensors, processors, memory, etc.) for monitoring the breath, calculating or otherwise determining the amount of NO to be delivered, and be in communication with other components of the NO delivery device (e.g. flow sensors, pressure sensors, valves, gas conduits, etc.) for delivering the gas comprising NO. The amount of NO per breath can be calculated and/or adjusted after every breath or can be calculated and/or adjusted at certain intervals such as every minute, every 10 minutes, every 10 breaths, every 100 breaths, etc.

In one or more embodiments, the iNO is not delivered to the patient every breath and at least one breath is skipped during the iNO therapy. The time period between individual pulses of gas comprising NO can vary or can be constant. In various embodiments, a maximum time period between pulses, a maximum average time period between pulses and/or a minimum pulse frequency may be provided.

Various situations can result in iNO being skipped in a particular breath. For example, an intermittent dosing regimen may be utilized in which the iNO is administered every n^{th} breath, with n being greater than 1. In various embodiments, n is about 1.01, about 1.1, about 1.2, about 1.3, about 1.4, about 1.5, about 1.6, about 1.7, about 1.8, about 1.9, about 2, about 2.5, about 3, about 4, about 5, about 6, about 7, about 8, about 9 or about 10. When n is not a whole number (e.g. 1.1 or 2.5), n can represent an average over multiple breaths. As an example, administering iNO every 2.5 breaths indicates that iNO is administered an average of 2 breaths out of every 5 breaths (i.e. 5/2=2.5). Similarly, administering iNO every 1.1 breaths indicates that iNO is administered an average of 10 breaths out of every 11 breaths (i.e. 11/10=1.1). Similar calculations can be performed for other intermittent dosing regimens where iNO is administered every n^{th} breath, with n being greater than 1.

In one or more embodiments, an intermittent dosing regimen may be utilized in which predetermined breaths are skipped. The skipping of predetermined breaths can be based on predetermined patterns such as skipping every other breath, skipping every third breath, skipping two consecutive breaths and delivering on the third breath, etc. The predetermined pattern can include delivering gas comprising NO on every n^{th} breath, such as having n be greater than 1, for example about 1.01, about 1.1, about 1.2, about 1.3, about 1.4, about 1.5, about 1.6, about 1.7, about 1.8, about 1.9, about 2, about 2.5, about 3, about 4, about 5, about 6, about 7, about 8, about 9 or about 10.

In one or more embodiments, one or more breaths is skipped in a certain time period. For example, 1, 2, 3, 4, 5, etc. breaths may be skipped every hour, every 30 minutes, every 15 minutes, every 10 minutes, every minute, every 30 seconds, etc. In some embodiments, as little as one breath is skipped during the entire iNO therapy. In other embodiments, multiple breaths are skipped during iNO therapy.

In one or more embodiments, an intermittent dosing regimen may be utilized in which random breaths are skipped. The random breath skipping can be determined according to a random number generator and/or can be based on current clinical conditions such as the patient's respiratory pattern, the patient's breathing rate, the amount of iNO that has been delivered to the patient, the patient's iNO prescription, etc., and/or can be based on settings for the NO delivery device such as a minimum pulse volume.

In one or more embodiments, the NO delivery device may have a minimum quantity of gas that can be delivered in a breath, such as a minimum pulse volume. This minimum quantity of gas can be set by the user or can be a minimum threshold value set by the specifications of the NO delivery device. In one or more embodiments, when the quantity of gas comprising NO to be delivered to the patient in a particular breath is less than the minimum quantity of gas per breath (e.g. minimum pulse volume), administration of the gas is skipped for that breath. In one or more embodiments, when the breath is skipped, a new quantity of gas per breath is calculated and/or the quantity of gas is carried over and is added to the amount of gas to be delivered in one or more subsequent breaths.

In addition to the exemplary situations described above, other situations that can result in one or more breaths being skipped during iNO therapy are also encompassed by the present disclosure. Such situations include, but are not limited to, skipped breaths or a pause in iNO therapy due to: changing or switching the drug cylinder or cartridge; NO delivery device purging; engagement with other devices or delivery systems such as LTOT, continuous positive airway pressure (CPAP), bilevel positive airway pressure (BPAP), etc.; NO delivery device alarm conditions such as apnea, empty drug cylinder/cartridge, empty battery, etc.; or NO delivery device fault condition(s).

In one or more embodiments, there is a maximum time period between successive pulses of the gas comprising NO. For example, the time period between successive pulses may vary or may be constant, but an upper limit may be provided that prevents too long of a period between successive pulses of gas. In exemplary embodiments, the maximum time period between successive pulses of gas comprises NO does not exceed about 30, about 25, about 20, about 15, about 14, about 13, about 12, about 11, about 10, about 9, about 8.5, about 8, about 7.5, about 7, about 6.5 or about 6 seconds.

In one or more embodiments, the maximum time period between successive pulses of the gas comprising NO is provided as a maximum number of breaths. In exemplary embodiments, the maximum number of consecutive skipped breaths does not exceed four, three, two or one breaths.

In one or more embodiments, the average time period between successive pulses of the gas comprising NO does not exceed a certain time period, such as not exceeding about 30, about 25, about 20, about 15, about 14, about 13, about 12, about 11, about 10, about 9, about 8.5, about 8, about 7.5, about 7, about 6.5 or about 6 seconds. Again, the time period between individual pulses can vary or can be the same.

In one or more embodiments, the average number of consecutive skipped breaths does not exceed about 3, about 2.5, about 2, about 1.5, about 1 or about 0.5 breaths.

In one or more embodiments, the frequency of pulse administration is provided as a number of pulses in a given time period, such as pulses per hour. For example, in one or more embodiments the patient is administered at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 700, about 750, about 800, about 850, about 900, about 950 or about 1,000 pulses of the gas comprising NO per hour.

Shorter durations may also be used, and these pulse frequencies can likewise be expressed in terms of pulses per minute or other time period. In one or more embodiments, the patient is administered at least about 5, about 5.1, about 5.2, about 5.3, about 5.4, about 5.5, about 5.6, about 5.7, about 5.8, about 5.9 about 6, about 6.1, about 6.2, about 6.3, about 6.4, about 6.5, about 6.6, about 6.7, about 6.8, about 6.9 about 7, about 7.1, about 7.2, about 7.3, about 7.4, about 7.5, about 7.6, about 7.7, about 7.8, about 7.9 about 8, about 8.1, about 8.2, about 8.3, about 8.4, about 8.5, about 8.6, about 8.7, about 8.8, about 8.9 about 9, about 9.5, about 10, about 10.5, about 11, about 11.5, about 12, about 12.5, about 13, about 13.5, about 14, about 14.5, about 15, about 16, about 17, about 18, about 19 or about 20 pulses per minute.

In one or more embodiments, the iNO is administered for a certain amount of time each day. For example, the iNO may be administered for at least about. 1 hour a day. In various embodiments, the iNO is administered for at least about 1, about 2, about 3, about 4, about 5, about 6, about 7, about 8, about 9, about 10, about 11, about 12, about 16, about 18 or about 24 hours a day.

In one or more embodiments, the iNO is administered for a certain treatment time. For example, the iNO may be administered for at least 2 days. In various embodiments, the iNO is administered for at least about 2, about 3, about 4, about 5, about 6 or about 7 days, or about 1, about 2, about 3, about 4, about 5, about 6, about 7 or about 8 weeks, or about 1, about 2, about 3, about 4, about 5, about 6, about 7, about 8, about 9, about 10, about 11, about 12, about 18 or about 24 months, or 1, 2, 3, 4 or 5 years.

In one or more embodiments, the patient is also receiving long-term oxygen therapy (LTOT). In various embodiments, the LTOT is administered for at least about 1, about 2, about 3, about 4, about 5, about 6, about 7, about 8, about 9, about 10, about 11, about 12, about 16, about 18 or about 24 hours a day. In various embodiments, the LTOT is administered at a dose of about 0.5 L/min to about 10 L/min, such as about 0.5, about 1, about 1.5, about 2, about 2.5, about 3, about 4, about 5, about 6, about 7, about 8, about 9 or about 10 L/min. The LTOT may be administered continuously or via pulses.

### EXAMPLES

### Example 1—Effect of Long-Term iNO Therapy on RV Function in Subjects with PH-IPF

### Study Design

This study was an exploratory, three-part, clinical study to assess the effect of pulsed iNO on functional pulmonary imaging parameters in subjects with PH-COPD on LTOT (Part 1) PH-IPF on LTOT (Part 2 and Part 3) (IK-7002-COPD-006; NCT02267655). The objective of this exploratory study was to examine the utility of high resolution computed tomography (HRCT) to measure changes in functional respiratory imaging parameters as a function of short term iNO administration using a pulsed NO delivery device in subjects with PH-IPF (Part 2 and Part 3) on LTOT. The primary endpoint in this exploratory study is the change from baseline in lobar blood volume at total lung capacity (TLC) after dosing with pulsed iNO (Part 1), iNO or Placebo (Part 2a) and after 4 weeks iNO treatment (Part 3b) as measured by HRCT.

The secondary endpoints of Part 2a (acute; placebo vs. iNO 75 mcg/kg IBW/hr) were change in Borg CR10 leg fatigue and dyspnea scale, changes in breathing questionnaire and changes in right ventricular and left ventricular function.

The secondary endpoints of Part 2b (chronic dosing) were change in 6MWT with Borg CR10 leg fatigue and dyspnea scale and SpO2, at the beginning and end of the 6MWT and symptoms evaluated using a questionnaire with after 4 weeks use of iNO at a dose of 75 mcg/kg IBW/hr and 2 weeks post discontinuation of iNO.

The secondary endpoints of Part 3b (chronic dosing) were change in 6MWT with Borg CR10 leg fatigue and dyspnea scale and SpO2, at the beginning and end of the 6MWT and symptoms evaluated using a questionnaire after 4 weeks use of iNO at a dose of 30 mcg/kg IBW/hr.

The safety endpoints in this study were:
a. Incidence and severity of treatment emergent adverse events (AEs), including those related to device deficiency;
b. Incidence of MetHb levels>7.0%;
c. New symptoms that may be due to rebound PH associated with a temporal acute withdrawal of investigational study drug (i.e., symptoms occurring within 20 minutes of acute withdrawal and including those associated with investigational medical device malfunction or failure): systemic arterial oxygen desaturation, hypoxemia, bradycardia, tachycardia, systemic hypotension, near-syncope, syncope, ventricular fibrillation, and/or cardiac arrest;
d. New or worsening symptoms of left heart failure or pulmonary edema; and
e. Any decrease in systemic oxygenation measured by oxygen saturation of arterial blood by pulse oximetry (SpO2), i.e., hypoxia or oxygen saturation decrease, deemed by the Investigator to be clinically significant.

This was an exploratory clinical study to evaluated the utility of HRCT to measure the pharmacodynamic effects of short term pulsed administration of iNO using a pulsed NO delivery device in subjects with PH-IPF (Part 2 and Part 3) on LTOT.

In Part 2b and Part 3b change in 6MWT with Borg CR10 leg fatigue and dyspnea scale and SpO2, at the beginning and end of the 6MWT, and a symptoms questionnaire were used to assess the effects of long term pulsed iNO administered using a pulsed NO delivery device in subjects with PH associated with IPF on LTOT.

In Part 2 of the study the subjects needed to have severe PH, therefore PH in Part 2 was defined as sPAP≥50 mm Hg by 2-D echocardiogram. In Part 3 PH was defined as sPAP≥35 mm Hg by echocardiogram (Part 3).

The initial protocol intended that 4 subjects would be enrolled in Part 2. However during the conduct of Part 2 of the trial, after enrollment of 2 subjects, it was noticed that the two IPF patients included both suffered from a sudden increase in PAP after discontinuation with the use of iNO at a dose of 75 mcg/kg IBW/hr. It was decided to temporarily stop recruitment. One of the 2 subjects completed 4 weeks of chronic use in Part 2b.

In the amendment a total of 2 subjects participated in Part 3. The dose in Part 3 was lower than in Part 2 and each subject was titrated to the optimum dose as determined by the investigator. The dose of iNO was lowered to prevent the sudden swings in PAP. The dose was monitored with a RHC in place. The investigator found that both the next 2 subjects could titrate to iNO at a dose of 30 mcg/kg IBW/hr safely. This dose was used in these 2 subjects in Part 3.

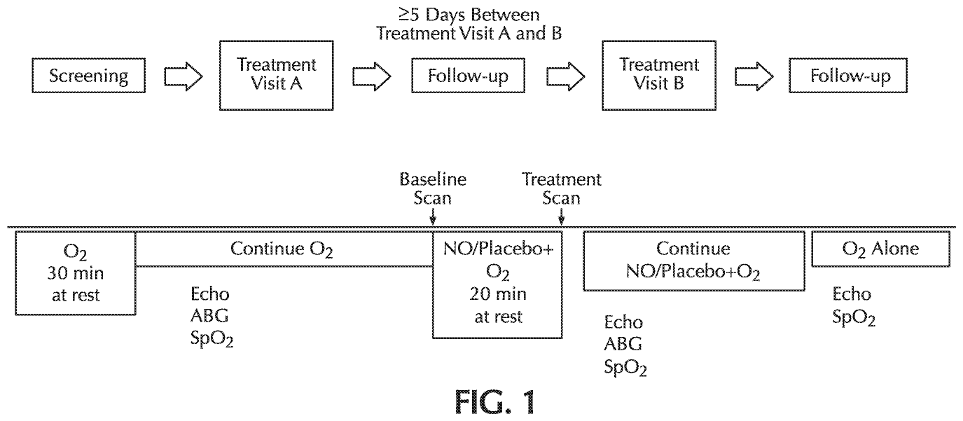

The 2 subjects enrolled in Part 2 were randomly assigned for Part 2a to 1 of 2 sequences to receive iNO utilizing the NO cylinder concentration (4,880 ppm) at a dose of 75 mcg/kg IBW/hr or placebo set at a dose of 75 mcg/kg IBW/hr. FIG. 1 shows the treatment visit schedule for Part 2a.

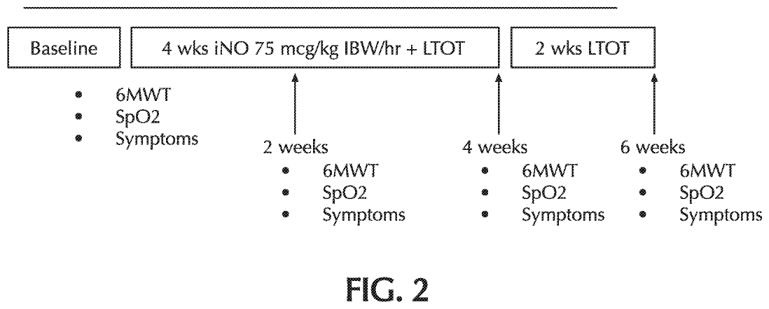

One patient from Part 2a entered into Part 2b. During Part 2b patient receive iNO utilizing NO cylinder concentration (4,880 ppm) at a dose of 75 mcg/kg IBW/hr during 4 weeks for at least 12 hours/day. The treatment visit schedule for Part 2b is summarized in FIG. 2.

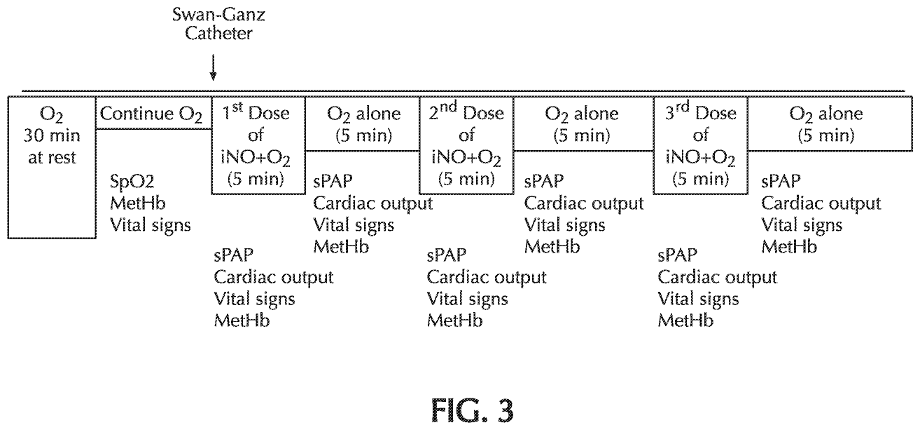

The 2 subjects enrolled in Part 3a each received three different doses of iNO utilizing NO cylinder concentration (4,880 ppm) at a dose of 5 mcg/kg IBW/hr, 10 mcg/kg IBW/hr and 30 mcg/kg IBW/hr, all with LTOT. For each dose, the change in PAP pressure and the change in cardiac output was evaluated by RHC. The investigator could decide after each dose to continue with the following dose or not. FIG. 3 shows the treatment visit dose titration details for Part 3a.

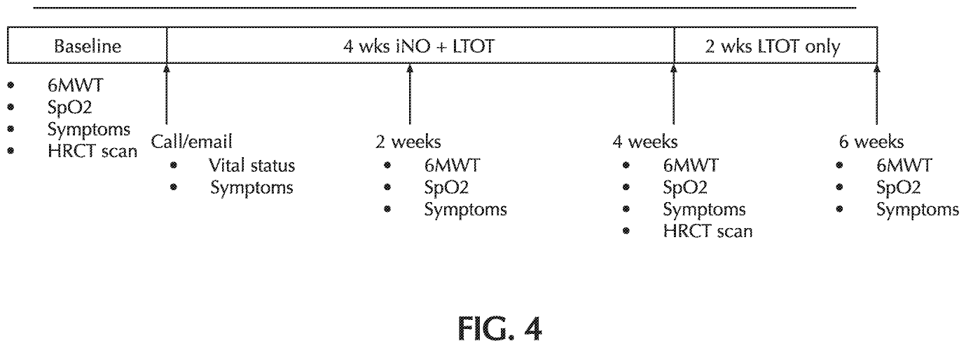

The 2 patients from Part 3a entered Part 3b. During Part 3b, patients received iNO utilizing NO cylinder concentration (4,880 ppm) at a dose of 30 mcg/kg IBW/hr. One subject did not tolerate the device and discontinued treatment after 2 weeks. FIG. 4 shows the treatment visit schedule for Part 3b.

The study population consisted of subjects ≥40 years, ≤80 years, with a confirmed diagnosis of IPF (Part 2 and Part 3) who are receiving LTOT and have PH. A total of 4 subjects were enrolled.

The study had the following inclusion criteria for Part 2 and Part 3:
a. Patients will have a diagnosis of IPF as determined by a responsible and experienced Respiratory physician and based on;
   i. HRCT: usual interstitial pneumonia
   ii. FVC: 50-90% of predicted FVC
b. PH defined as sPAP≥50 mm Hg by echocardiogram (Part 2) and sPAP≥35 mm Hg by echocardiogram or right heart catheterization (Part 3). If in Part 3a Screening Visit and Treatment Visit are performed on the same day documented results by echocardiogram or RHC from within 12 months prior to the Screening Visit should be available to evaluate eligibility.
c. Age≥40 years
d. Receiving LTOT for ≥3 months
e. Females of childbearing potential must have a negative pre-treatment urine pregnancy test
f. Signed informed consent prior to the initiation of any study mandated procedures or assessments
g. BMI≤35 (Part 3 only)

The study had the following key exclusion criteria for Part 2 and Part 3. Subjects who meet any of the following criteria were not eligible for enrollment:
a. Patients with a current IPF exacerbation or exacerbation within the past 30 days.
b. Clinically significant valvular heart disease that may contribute to PH, including mild or greater aortic valvular disease (aortic stenosis or regurgitation) and/or moderate or greater mitral valve disease (mitral stenosis or regurgitation), or status post mitral valve replacement
c. Use within 30 days of screening or current use of approved specific PH medications (ERA or PDE-5 inhibitor, or oral, inhaled, subcutaneous, or intravenous prostacyclin or a prostacyclin analog)
d. Use of investigational drugs or devices within 30 days prior to enrollment into the study
e. Any underlying medical or psychiatric condition that, in the opinion of the Investigator, makes the subject an unsuitable candidate for the study

### Results

As can be seen from the above description, patients with PH-IPF were put on acute and chronic treatment with iNO. During the chronic phase, the vasodilation and the hemodynamics were assessed. During the chronic phase, the focus was exercise capacity. Both the acute and chronic phases evaluated iNO doses of 30 and 75 mcg/kg IBW/hr.

Table 1 below shows the acute effect of iNO on blood vessel volume as well as sPAP.

TABLE 1
Changes in Blood Vessel Volume and sPAP in PH-IPF Subjects

| | Patient 1 | Patient 2 | Patient 3 | Patient 4 |
|---|---|---|---|---|
| iNO Dose | 75 | 75 | 30 | 30 |
| (mcg/kg IBW/hr) | | | | |
| Acute change | 14.0 ± 4.7 | 34.2 ± 7.6 | 2.8 ± 3.0 | 10.1 ± 3.4 |
| in blood vessel | | | | |
| volume (%) | | | | |
| Acute Change | −9.3 | −9.7 | −14.3 | −23.3 |
| in sPAP (%) | | | | |

As can be seen from Table 1, the increase in blood vessel volume is much higher for the iNO dose of 75 mcg/kg IBW/hr dose compared to the iNO dose of 30 mcg/kg IBW/hr. However, the effect on sPAP is similar or skewed towards the lower iNO dose of 30 mcg/kg IBW/hr.

Table 2 below shows the TAPSE results from two PH-IPF subjects in this trial. Subject 1 received pulsed iNO at a dose of 75 mcg/kg IBW/hr for 4 weeks, and Subject 3 received pulsed iNO at a dose of 30 mcg/kg IBW/hr for 4 weeks.

TABLE 2
Changes in TAPSE in PH-IPF Subjects
During Chronic iNO Therapy

TAPSE

| CRF ID | Baseline | 4 Week | Increase | % Change |
|---|---|---|---|---|
| Subject 1 | 14 | 17 | 3 | 21% |
| Subject 3 | 25 | 28 | 4 | 12% |
| Average | 20 | 23 | 3 | 15% |

As can be seen from Table 2, these results show that the long-term pulsed iNO therapy increased TAPSE in both PH-IPF subjects. This increase in TAPSE indicates an improvement in RV function.

### Example 2—Effect of Long-Term iNO Therapy on RV Function in Subjects with PH-COPD

This study is an open label Phase 1 study of iNO therapy in subjects with PH-COPD (PULSE-COPD-007; NCT03135860). The primary outcome of this study is the change in lobar blood volume at total lung capacity with iNO and the change in lobar blood volume with iNO after 4 weeks of treatment with iNO as measured by HRCT.

Subjects had a confirmed diagnosis of COPD by the Global initiative for chronic Obstructive Lung Disease (GOLD) criteria. Subjects also had sPAP≥38 mm Hg as measured by echocardiogram, a post-bronchodilatory FEV1/FVC<0.7 and a FEV1<60% predicted. All subjects were at least 40 years old and were current or former smokers with at least 10 pack-years of tobacco cigarette smoking before study entry. All subjects also had been receiving LTOT for at least 3 months for at least 10 hours per day.

The PH-COPD subjects received pulsed iNO therapy for 4 weeks for at least 12 hours/day. The iNO was administered utilizing a 4,880 ppm NO cylinder concentration.

Table 3 below shows the TAPSE results from four PH-COPD subjects in this trial. These subjects were diagnosed with PH-COPD and received 4 weeks of treatment with iNO at a dose of 30 mcg/kg IBW/hr. The results verify the increase in TAPSE which correlates to RV function.

TABLE 3
Changes in TAPSE in PH-COPD Subjects
During Chronic iNO Therapy

TAPSE

| CRF ID | Baseline | 4 Week | Increase | % Change |
|---|---|---|---|---|
| Subject 1 | 11 | 18 | 7 | 67% |
| Subject 6 | 23 | 28 | 5 | 22% |
| Subject 7 | 16 | 18 | 2 | 13% |
| Subject 12 | 14 | 14 | 0 | 0% |
| Average | 16 | 20 | 4 | 25% |

As can be seen from Table 3, these results show that the long-term pulsed iNO therapy increased TAPSE in three subjects, and TAPSE did not change in the fourth subject. This increase in TAPSE indicates an improvement in RV function for the three subjects and a maintenance in RV function for the fourth subject. Overall, the average increase in TAPSE of 25% across all four subjects shows that iNO therapy improves and/or maintains RV function.

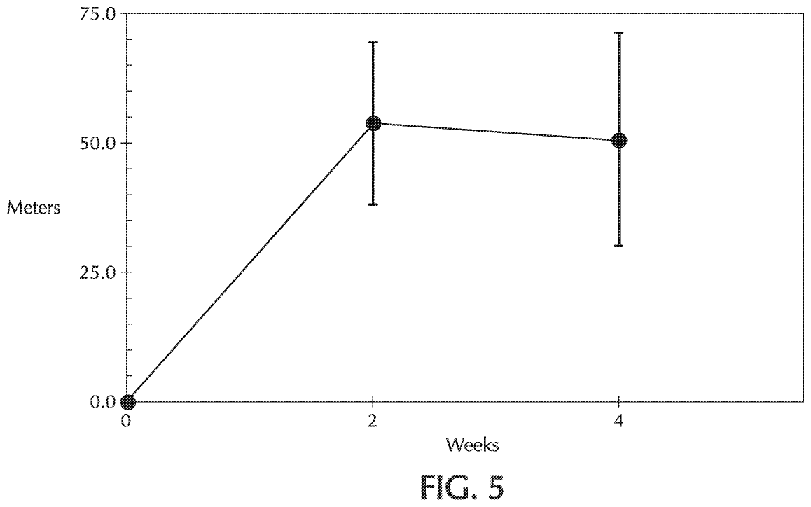

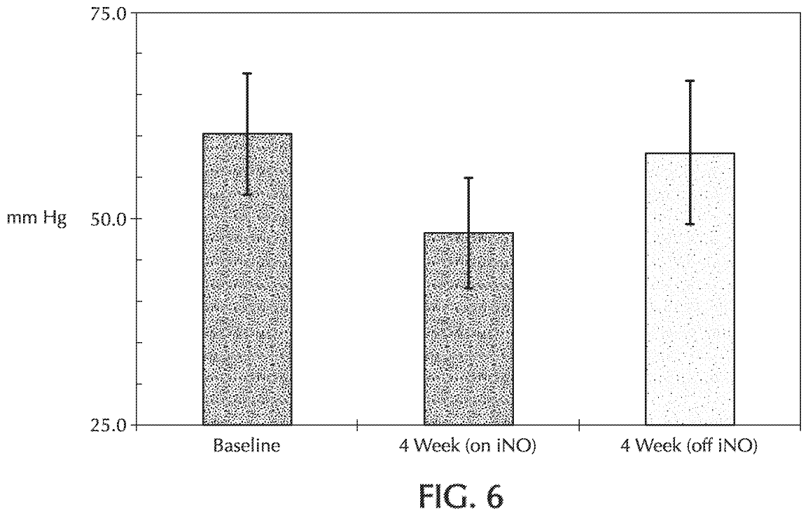

A further analysis was performed of seven PH-COPD subjects that completed 4 weeks of treatment with iNO at a dose of 30 mcg/kg IBW/hr. A summary of the baseline, acute and chronic parameters for these patients in shown in Table 4 below. The 6MWD and sPAP results are also presented in FIGS. 5 and 6, respectively.

TABLE 4
Acute Change in Blood Vessel Volume and Chronic
Changes in sPAP and 6MWD in PH-COPD Subjects

| | #1 | #2 | #3 | #4 | #5 | #6 | #7 |
|---|---|---|---|---|---|---|---|
| Subject ID No. | 001 | 007 | 006 | 012 | 010 | 013 | 014 |
| Age (yrs)/Sex | 52/M | 62/M | 59/F | 60/M | 62/M | 72/M | 79/M |
| Compliance (hrs/day) | 19.9 | 9.9 | 9 | 23.2 | 11.9 | 16.1 | 17.2 |
| Acute change | 6.2 ± 1.6 | 3.3 ± 2.1 | 6.6 ± 4.5 | 9.7 ± 3.5 | −1.0 ± 4.0 | N/A* | 2.7 ± 0.4 |
| in Blood | | | | | | | |
| Vessel Volume % | | | | | | | |

Chronic change in sPAP (mm Hg)

| | | | | | | | |
|---|---|---|---|---|---|---|---|
| Baseline | 94 | 47 | 55 | 78 | 40 | 46 | 62 |
| 4 weeks iNO | 69 | 37 | 40 | 74 | 30 | 34 | 54 |
| Change sPAP | −25 | −10 | −15 | −4 | −10 | −12 | −8 |
| (mm Hg) | | | | | | | |
| % Change | −27% | −21% | −27% | −5% | −25% | −26% | −13% |

Chronic change in 6MWD (meters)

| | | | | | | | |
|---|---|---|---|---|---|---|---|
| Baseline | 200 | 184 | 478 | 80 | 470 | 343 | 142 |
| 2 weeks iNO | 335 | 263 | 493 | 115 | 495 | 400 | 173 |
| Change from | +135 | +79 | +15 | +35 | +25 | +57 | +32 |
| Baseline | | | | | | | |
| 4 weeks iNO | 335 | 195 | 480 | 77 | 498 | 423 | 242 |
| Change from | +135 | +11 | +2 | −3 | +28 | +80 | +100 |
| Baseline | | | | | | | |

*Method error during testing

iNO 30 mcg/kg/IBW resulted in a significant increase in the 6MWD (FIG. 5) and decrease in sPAP as measured by echocardiogram (FIG. 6). As shown in FIG. 5, the change in 6MWD after 2 weeks of iNO therapy is +53.9 meters (p=0.02). Similarly, the change in 6MWD after 4 weeks of iNO therapy is +50.7 meters (p=0.04). In the literature, 27-54 meter improvements in 6MWD are considered clinically significant as measured by patient perceptions of improvement.

As shown in FIG. 6, the sPAP at baseline was 60.3 mm Hg. After 4 weeks of iNO therapy, the sPAP was 48.3 mm Hg [12.0 mm Hg drop; 19.9% drop] (p=0.02). 4 weeks after iNO therapy was discontinued, the sPAP increased to 58.0 mm Hg.

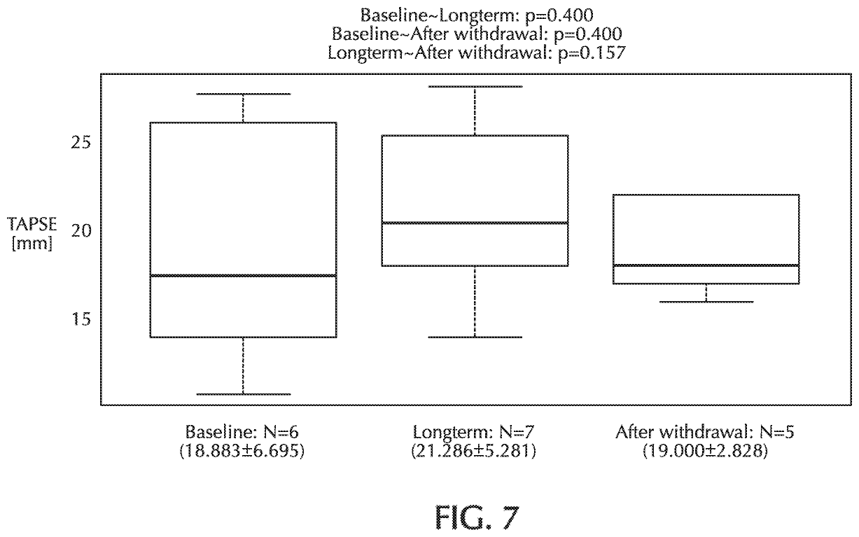

The decrease in sPAP correlated with a trend in the improvement in RV function as measure by TAPSE, as shown in FIG. 7. The baseline TAPSE was 18.8 (N=6), the TAPSE after chronic iNO therapy was 21.3 (N=7) and the TAPSE after iNO therapy was discontinued was 19.0 (N=5). These results further confirm that iNO therapy improves and/or maintains RV function.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

1. A method of maintaining or improving right ventricular function, the method comprising administering to a patient in need thereof an effective amount of inhaled nitric oxide (iNO) for at least 2 days.

2. The method of claim 1, wherein the patient has pulmonary hypertension.

3. The method of claim 2, wherein the pulmonary hypertension comprises one or more of pulmonary arterial hypertension (WHO Group I), pulmonary hypertension associated with left heart disease (WHO Group 2), pulmonary hypertension associated with lung disease and/or chronic hypoxemia (WHO Group 3), chronic thromboembolic pulmonary hypertension (WHO Group 4) or pulmonary hypertension with unclear multifactorial mechanisms (WHO Group 5).

4. The method of claim 1, wherein the patient has pulmonary arterial hypertension (WHO Group I).

5. The method of claim 1, wherein the patient has WHO Group 3 pulmonary hypertension associated with idiopathic pulmonary fibrosis (IPF) or chronic obstructive pulmonary disease (COPD).

6. The method of claim 1, wherein the patient has been placed on a lung transplant waiting list.

7. The method of claim 1, wherein the patient has received a lung transplant.

8. The method of claim 1, wherein the patient has a ventilation-perfusion (V/Q) mismatch.

9. The method of claim 1, wherein the iNO is administered for at least 1 week.

10. The method of claim 1, wherein the iNO is administered for at least 2 weeks.

11. The method of claim 1, wherein the iNO is administered for at least 4 weeks.

12. The method of claim 1, wherein the iNO is administered for at least 3 months.

13. The method of claim 1, wherein the iNO is administered for at least 6 hours a day.

14. The method of claim 1, wherein the iNO is administered for at least 12 hours a day.

15. The method of claim 1, wherein the effective amount of iNO is in the range of about 10 to about 300 micrograms NO per kilogram ideal body weight per hour (mcg/kg IBW/hr).

16. The method of claim 1, wherein the effective amount is in the range of about 30 to about 75 mcg/kg IBW/hr.

17. The method of claim 1, wherein maintaining or improving right ventricular function comprises maintaining or improving one or more of the following parameters: right ventricular fractional area change (RVFAC), tricuspid annular motion, tricuspid annular plane systolic excursion (TAPSE), systolic pulmonary artery pressure (sPAP), tricuspid annular systolic velocity (TASV), and Tei index.

18. The method of claim 1, wherein the administration of iNO provides an average increase in TAPSE in a group of patients after 4 weeks of iNO administration of at least 1 millimeter (mm).

19. The method of claim 1, wherein the administration of iNO provides an average increase in TAPSE in a group of patients after 4 weeks of iNO administration of at least 2 mm.

20. The method of claim 1, wherein the administration of iNO provides an average increase in TAPSE in a group of patients after 4 weeks of iNO administration of at least 5%.

21. The method of claim 1, wherein the administration of iNO provides an average increase in TAPSE in a group of patients after 4 weeks of iNO administration of at least 10%.

22-59. (canceled)
